(19) United States
(12) Patent Application Publication
(10) Pub. No.: US 2006/0069347 A1
(43) Pub. Date: Mar. 30, 2006

(54) Safety syringe sealing system and method of use

(76) Inventor: Quent D Besing, Eureka, MO (US)

Correspondence Address: Mallinckrodt Inc., 675 McDonnell Boulevard, PO Box 5840, St. Louis, MO, 63134, US

(21) Appl. No.: 10/521,643

(22) PCT Filed: Aug. 5, 2003
(86) PCT No.: PCT/US03/24510

§ 371 Date: Jan. 11, 2005

Related U.S. Application Data
(60) Provisional application No. 60/403,775, filed on Aug. 15, 2002.

(51) Int. Cl. A61M 5/00; A61M 5/32

(52) U.S. Cl. 604/110; 604/198

(57) ABSTRACT

A safety syringe sealing system and method of use is disclosed. The safety syringe sealing system includes the combination of a safety syringe and a plug. The safety syringe includes a barrel, a needle, a plunger and a needle sheath. The needle sheath is movable between a retracted position where the needle is exposed, to an extended position where the needle is protected, the needle sheath defining an open end. There is a plug that is engageable with the open end of the needle sheath when the needle sheath is in the extended position. The sealing system can also include a pharmaceutical pig, having a base and cap, where the base and cap define a hollow center section that is capable of receiving the safety syringe. Therefore, the safety syringe, having a plugged needle sheath, may be sealed within the pharmaceutical pig to prevent leakage and inadvertent exposure.

### BACKGROUND OF INVENTION

The Needlestick Prevention Act was enacted by the United States Congress and went into effect on Nov. 6, 2000 with a requirement of compliance by Apr. 18, 2001. The U.S. Congress directed the Occupational Safety and Health Administration, hereinafter “OSHA”, to administer the Act. In accordance therewith, OSHA then promulgated revised regulations relating to the control of bloodborne pathogens (Bloodborne Pathogens Standard, 29 C.F.R. Section 1910.1030). This revised Bloodborne Pathogens Standard applies to all occupational exposure to blood or other potentially infectious material. The Needlestick Prevention Act is an attempt to prevent the 600,000 to 800,000 inadvertent exposures to bloodborne pathogens that occur each year. Needlesticks occur at a rate of 300 inadvertent exposures per 100 healthcare treatment beds each year. The Act requires healthcare facilities under the jurisdiction of OSHA to use safer medical devices, such as sharps with engineered sharps injury protection and needleless systems. This Act and other OSHA directives require an annual review of a facility's exposure control plan and the use of safer medical devices to help reduce needlesticks and other sharps injuries. The Needlestick Safety and Prevention Act, as well as other state rules and regulations, have encouraged the development of “safety syringes”. The purpose of the safety syringe is to keep the tip of the needle or fluids in the syringe from inadvertently contacting anyone before and after a medication has been administered to a patient.

There are a number of safety syringe designs such as those that have retractable or sheathed needles. One particular type of safety syringe includes an extendable, sliding sheath design where the sheath moves from a retracted position to an extended position that surrounds the needle after it has been used. Examples include the Monoject® safety syringe, manufactured by Kendall-LTP, also know as The Ludlow Company LP, a business of Tyco International Ltd., having a place of business at Two Ludlow Park Drive, Chicopee, Mass. 01022, “The Safety-Lok™” from Becton-Dickinson and Company, having a place of business at 1 Becton Drive, Franklin Lakes, N.J. 07417, the Gettig Guard® syringe from Gettig Technologies, Inc., having a place of business at 1 Streamside Place, Spring Mills, Pa. 16875 and the Univec Sliding Sheath® syringe from Univec Inc., having a place of business at 22 Dubon Court, Farmingdale, N.Y., among others.

Safety syringes with an extendable sheath design are also disclosed in: U.S. Pat. No. 4,994,045, issued to Ranford on Feb. 19, 1991, which is incorporated herein by reference; U.S. Pat. No. 4,998,924, issued to Ranford on Mar. 12, 1991, which is incorporated herein by reference; U.S. Pat. No. 4,743,233, issued to Schneider on May 10, 1988, which is incorporated herein by reference; U.S. Pat. No. 5,403,287, issued to Talonn et al. on Apr. 4, 1995, which is incorporated herein by reference; U.S. Pat. No. 5,163,916, issued to Sunderland on Nov. 17, 1992, which is incorporated herein by reference; U.S. Design Pat. No. 313,470, issued to Talonn et al. on Jan. 1, 1991, which is incorporated herein by reference; and U.S. Design Pat. No. 344,355, issued to Talonn et al. on Feb. 15, 1994, which is incorporated herein by reference.

A radiopharmacy typically dispenses a liquid radiopharmaceutical in a conventional syringe, which is then placed in a pharmaceutical pig for transport to a medical facility. The pharmaceutical pig reduces unwanted exposure from the radioactive material and protects the conventional syringe from damage during transport. After delivery, the pharmaceutical pig is opened, the conventional syringe is removed and the liquid radiopharmaceutical is administered to a patient. The used syringe is put back in the pharmaceutical pig and returned to the radiopharmacy for disposal. Residual blood and liquid radiopharmaceutical can leak from the conventional needle to contaminate the inside of the pharmaceutical pig and create an increased exposure risk. Moreover, before and during the securing of this contaminated conventional needle within the pharmaceutical pig, accidental exposure to bloodborne pathogens can occur. In fact, the “danger zone” is specifically defined as the time period from the point of patient injection to the point of disposing of the syringe. Fifty percent (50%) of all inadvertent exposures to bloodborne pathogens occur after the patient is injected and twenty percent (20%) occur when disposing of the syringe into a sharps container.

Some radiopharmacies are independently owned and others are owned and operated in nationwide networks by Cardinal Health, Inc., having a place of business at 7000 Cardinal Place, Dublin, Ohio 430017 and Mallinckrodt Inc., a business of Tyco International, Ltd., having a place of business at 15 Hampshire Street, Mansfield, Mass. 02048. Pharmaceutical pigs currently used with syringes are elongate devices sized to enclose a single conventional syringe that holds a dose for a single patient. Conventional pharmaceutical pigs are available from Biodex Medical Systems, Inc., having a place of business at 20 Ramsay Road, Shirley, N.Y. 11967-0702 and are available from Cardinal Healthcare Ltd.

### SUMMARY OF INVENTION

In one aspect of this present invention, a safety syringe sealing system is disclosed. The system includes a combination safety syringe and plug. The safety syringe includes a barrel, a needle, a plunger and a tubular needle sheath. The needle sheath is movable between a retracted position where the needle is exposed, to an extended position where the needle is protected and the needle sheath defines an open end. In the preferred embodiment, the plug engages the outer circumference of the open end of the tubular needle sheath. In an alternative embodiment, the plug engages the inner circumference of the open end of the tubular needle sheath.

In another aspect of this present invention, a safety syringe sealing system is disclosed. The system includes a combination pharmaceutical pig, safety syringe and plug. The safety syringe includes a barrel, a needle, a plunger and a needle sheath. The needle sheath is movable between a retracted position where the needle is exposed, to an extended position where the needle is protected and the needle sheath defines an open end. The plug is engageable with the open end of the needle sheath when the needle sheath is in the extended position. As mentioned above, the plug may engage the outer circumference or the inner circumference as well as the open end of the tubular needle sheath. The pharmaceutical pig includes a base and cap with the base and cap defining a hollow center section that is capable of receiving the safety syringe.

In yet another aspect of this present invention, a method for sealing a safety syringe is disclosed. The method includes sliding a needle sheath longitudinally from a retracted position where a needle is exposed to an extended position where the needle is protected. The needle sheath defining an open end and engaging a plug with the open end of the needle sheath when the needle sheath is in the extended position. In the preferred method, the plug engages the outer circumference of the open end of the tubular needle sheath. In an alternative method, the plug engages the inner circumference of the open end of the tubular needle sheath.

In still another aspect of this present invention, a method for plugging a safety syringe is disclosed. The method includes sliding a tubular needle sheath longitudinally from a retracted position where a needle is exposed to an extended position where the needle is protected. The tubular needle sheath defining an open end, locking the tubular needle sheath into the extended position, and engaging a plug, having a cylindrical portion with at least one flange extending radially therefrom with the open end of the needle sheath in the extended position. As mentioned above, the plug may engage the outer circumference or the inner circumference of the needle sheath.

In another aspect of this present invention, a method for sealing a safety syringe is disclosed. This method includes inserting a plug in a hollow center section of a base of a pharmaceutical pig having a cap, sliding a needle sheath longitudinally from a retracted position to an extended position where the needle is protected with the needle sheath defining an open end, inserting the safety syringe with the needle sheath into the hollow center section of the base of the pharmaceutical pig, and engaging the plug with the open end of the needle sheath in the extended position.

In yet another aspect of this present invention, a method for sealing a safety syringe is disclosed. The method includes inserting a plug in a hollow center section of a base of a pharmaceutical pig having a cap, sliding a needle sheath longitudinally from a retracted position to an extended position where the needle is protected with the needle sheath defining an open end, engaging the plug with the open end of the needle sheath in the extended position, and inserting the safety syringe with the needle sheath into the hollow center section of the base of the pharmaceutical pig. Again, the plug may engage the outer circumference of the needle sheath or it may engage the inner circumference of the needle sheath.

These are merely some of the innumerable illustrative aspects of this present invention and should not be deemed an all-inclusive listing. These and other aspects will become apparent to those skilled in the art in light of the following disclosure and accompanying drawings.

### BRIEF DESCRIPTION OF DRAWINGS

For a better understanding of the present invention, reference may be made to the accompanying drawings in which:

FIG. 1 is a sectional view of a medium size safety syringe, a barrel, a needle, a plunger and a needle sheath, wherein the needle sheath is movable between a retracted position where the needle is exposed to an extended position where the needle is protected and the needle sheath defining an open end when the extendable sheath is fully extended. In FIG. 1, the needle sheath is in the retracted position.

FIG. 2 is a sectional view of the safety syringe with the needle sheath in the fully extended position. The needle sheath and the plug are not engaged. The safety syringe in FIG. 2 is an example of a medium size safety syringe.

FIG. 3 is a sectional view of the safety syringe with the needle sheath in the fully extended position. The needle sheath and the plug are engaged. The safety syringe in FIG. 3 is an example of a medium size safety syringe.

FIG. 4 is an enlarged side elevational view of the preferred embodiment of the plug of FIGS. 2 and 3.

FIG. 5 is an enlarged top view of the preferred embodiment of the plug of FIG. 4.

FIG. 6 is an enlarged bottom view of the preferred embodiment of the plug of FIG. 4.

FIG. 7 is an enlarged sectional view of the preferred embodiment of the plug along the line 7-7 of FIG. 5.

FIG. 8 is a sectional view of the safety syringe with the needle sheath in the fully extended position. A side elevational view of the preferred embodiment of the plug is shown. The needle sheath and the plug are not engaged. The safety syringe in FIG. 8 is an example of a small size syringe.

FIG. 9 is a sectional view of the safety syringe with the needle sheath in the fully extended position. The needle sheath and the plug are engaged. The safety syringe in FIG. 9 is an example of a small size syringe.

FIG. 10 is a sectional view of the safety syringe with the needle sheath in the fully extended position. The needle sheath and the plug are not engaged. The safety syringe in FIG. 10 is an example of a large size syringe.

FIG. 11 is a sectional view of the safety syringe with the needle sheath in the fully extended position. The needle sheath and the plug are engaged. The safety syringe in FIG. 11 is an example of a large size syringe.

FIG. 12 is a sectional view of an assembled pharmaceutical pig, with the safety syringe and plug as shown in FIG. 11 positioned inside the pharmaceutical pig.

FIG. 13 is a sectional view of the safety syringe with the needle sheath in the fully extended position. The needle sheath and an alternative embodiment of the plug are not engaged.

FIG. 14 is a sectional view of a safety syringe with the needle sheath in the fully extended position. The needle sheath and an alternative embodiment of the plug are engaged.

FIG. 15 is a sectional view of an assembled pharmaceutical pig, with the safety syringe and plug as shown in FIG. 14 positioned inside the pharmaceutical pig.

### DETAILED DESCRIPTION

In the following detailed description, numerous specific details are set forth in order to provide a thorough understanding of the invention. However, it will be understood by those skilled in the art that the present invention may be practiced without these specific details. For example, the invention is not limited in scope to a particular brand of safety syringe, plug or pharmaceutical pig depicted in the Drawings.

Safety syringes are produced in a wide variety of different sizes including, but not limited to: 1 cubic centimeter (0.06 cubic inches); 3 cubic centimeters (0.18 cubic inches); 5 cubic centimeters (0.31 cubic inches); 6 cubic centimeters (0.37 cubic inches); 10 cubic centimeters (0.61 cubic inches); and 12 cubic centimeters (0.73 cubic inches). An illustrative, but nonlimiting, example of a safety syringe is the Monoject® safety syringe, manufactured Kendall-LTP, also know as The Ludlow Company LP, a business of Tyco International Ltd., having a place of business at Two Ludlow Park Drive, Chicopee, Mass. 01022. However, other types of safety syringes may be utilized with the present invention provided they have a needle sheath with an open end when the needle sheath is in the extended position. These safety syringes include, but are not limited to “The Safety-Lok™” from Becton-Dickinson and Company, the Gettig Guard® syringe from Gettig Technologies, Inc. and the Univec Sliding Sheath® syringe from Univec Inc.

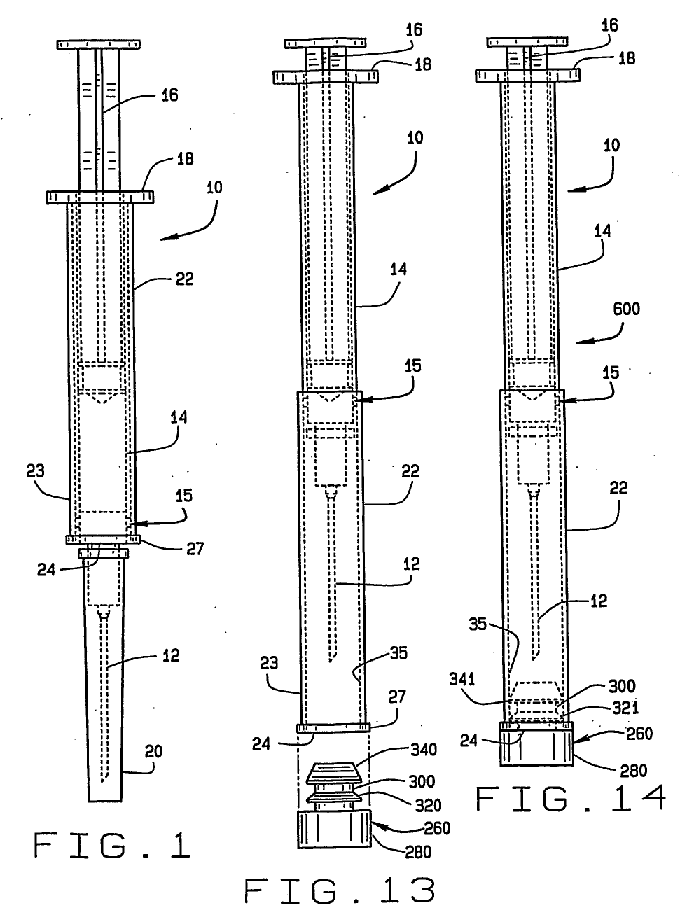

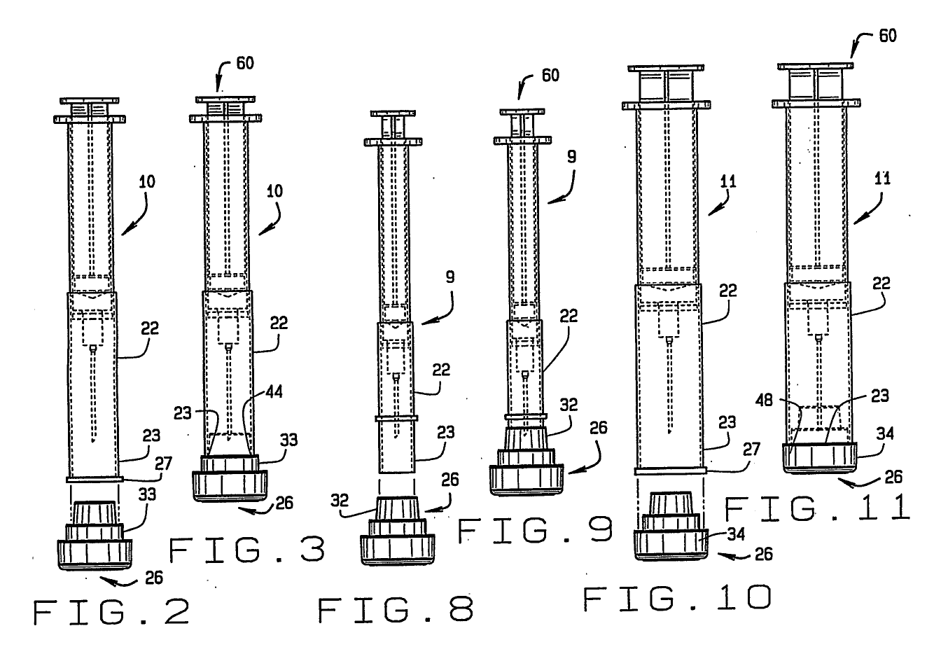

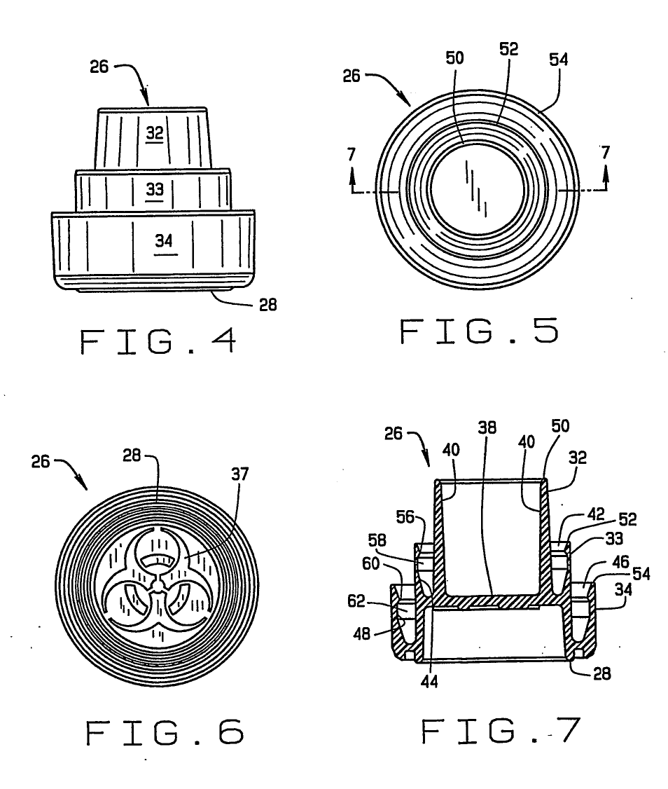

To illustrate this invention, a Monoject® safety syringe having a 3 cubic centimeters (0.18 cubic inches) capacity was selected from the wide field of suitable safety syringes and has been generally identified by the numeral 10 as shown in FIGS. 1-3. The safety syringe 10 may sometimes hereinafter be simply referred to as the medium size safety syringe. A Monoject® safety syringe having a 1 cubic centimeter (0.06 cubic inches) has also been selected to illustrate that the invention can be used with safety syringes of different sizes. The 1 cubic centimeter (0.06 cubic inches) capacity safety syringe has been generally identified by the numeral 9 as shown in FIGS. 4 and 5. The safety syringe 9 may sometimes hereinafter be simply referred to as the small safety syringe. To further illustrate that this invention can be used with a wide variety of different safety syringes, a 6 cubic centimeters (0.37 cubic inches) Monoject® safety syringe has also been selected and is generally identified by the numeral 11. The 6 cubic centimeters (0.37 cubic inches) safety syringe 11 may sometimes hereinafter be simply referred to as the large safety syringe, which is best seen in FIGS. 10 and 11. Because the small safety syringe 9, the medium safety syringe 10 and the large safety syringe 11 have many similar components, similar numerals will be used to identify these common components throughout the drawings.

As shown in FIG. 1, the medium safety syringe 10 includes a needle 12, shown in phantom, a barrel 14, which is also shown in phantom, a plunger 16 and finger grips 18, which are also known as wings. The finger grips 18 may be hexagonal, circular or polygonal and may fully or partially surround the barrel 14. There is a removable needle cover 20 that surrounds and protects the needle 12. There is an extendable, tubular sheath 22 that is in the retracted position and surrounds the barrel 14. The extendable, tubular sheath 22 includes an open end 24 having an outer circumference 23.

FIG. 2 is a sectional view of the medium safety syringe 10 with the extendable, tubular sheath 22 in the fully extended position. A side elevational view of the preferred embodiment of the plug 26 is shown. The extendable, tubular sheath 22 and the plug 26 are not engaged. Prior to administration of a radiopharmaceutical to a patient, the medium safety syringe 10 is placed in a radiation/injection shield (not shown) and the needle cover 20, as shown in FIG. 1, is then removed. After administration of the radiopharmaceutical to the patient, the extendable, tubular sheath 22 is moved from the fully retracted position of FIG. 1 to the fully extended position of FIG. 2, wherein the needle 12 is fully surrounded by the extendable, tubular sheath 22. The extendable, tubular sheath 22 is then locked and prevented from returning to the retracted position with a locking mechanism, which is generally indicated by numeral 15, by rotating the extendable, tubular sheath 22 about the barrel 14 of the medium safety syringe 10. A locking mechanism 15 is described in previously referenced U.S. Pat. No. 5,403,287, issued to Talonn et al. on Apr. 5, 1995, which is again incorporated herein by reference.

FIG. 3 is a sectional view of the medium safety syringe 10 with the extendable, tubular sheath 22 in the fully extended position. The extendable, tubular sheath 22 and the plug 26 are engaged. The outer circumference 23 of the extendable, tubular sheath 22 has engaged the plug 26.

The preferred embodiment of a plug 26 will be described with reference to FIGS. 4 and 7. This plug 26 can be of a wide variety of shapes and sizes, which operate to close the open end 24 of the extendable, tubular sheath 22. The preferred, but nonlimiting, embodiment of the plug 26 includes an end portion 28, a first cylindrical portion 32, a second cylindrical portion 33 and a third cylindrical portion 34. Preferably, but not necessarily, the first cylindrical portion 32 has the smallest diameter and the greatest height. The first cylindrical portion 32 can be utilized with a small safety syringe 9 having a relatively small volume, e.g., 1 cubic centimeter (0.06 cubic inches). Moreover, preferably but not necessarily, the second cylindrical portion 33 has a medium diameter and a medium height. The second cylindrical portion 33 can be utilized with a medium safety syringe 10 having a medium volume, e.g., 3 cubic centimeters (0.18 cubic inches). Finally, preferably but not necessarily, the third cylindrical portion 34 has the largest diameter and the lowest relative height. The third cylindrical portion 34 can be utilized with a large safety syringe 11 having a relatively large volume, e.g., 6 cubic centimeters (0.37 cubic inches). More than three sizes of safety syringes and associated portions may be used in a single plug 26 and there is no necessity that the height of each cylindrical portion 32, 34 and 36 vary in relation to other cylindrical portions in any direction positive or negative.

Referring now to FIGS. 4, 6 and 7, a base 28 for the plug 26 is shown. Preferably, but not necessarily, a biohazard symbol 37 is formed in the base 28, as shown in FIG. 6. The first cylindrical portion 32 defines a first u-shaped receptacle 38, which has a first inner circumferential wall 40, as shown in FIG. 7, that is sized to engage the outer circumference 23 of a small safety syringe 9, as better seen in FIGS. 8 and 9. The second cylindrical portion 33 forms a second u-shaped receptacle 42 which has a second inner circumferential wall 44, as shown in FIG. 7, that is sized to engage the outer circumference 23 of a medium safety syringe 10, as better seen in FIGS. 2 and 3. The third cylindrical portion 34 forms a third u-shaped receptacle 46, which has a third inner circumferential wall 48, as shown in FIG. 7, that is sized to engage the outer circumference 23 of a large safety syringe 11, as better seen in FIGS. 10 and 11. Those skilled in the art will recognize that the size and shape of the cylindrical portions 32, 33 and 34 can be arranged to accommodate a wide variety of safety syringes having different sizes and the terms small, medium and large are used merely to illustrate that syringes of different sizes can be sealed with a single plug 26.

The first cylindrical portion 32 defines a first circular lip 50 which is sized to receive the extendable, tubular sheath 22 of a small safety syringe 9, as better seen in FIG. 9. The second cylindrical portion 33 defines a second circular lip 52 which is sized to receive the extendable, tubular sheath 22 of the medium safety syringe 10, as better seen in FIG. 3. The third cylindrical portion 34 defines a third circular lip 54 which is sized to receive the extendable, tubular sheath 22 of a large safety syringe 11, as better seen in FIG. 11. Those skilled in the art will recognize that the respective diameters of the circular lips 50, 52 and 54 can be arranged to accommodate a wide variety of different size syringes and the terms small, medium and large are used merely to illustrate that syringes of various shapes and sizes can be used with a single plug.

The first inner circumferential wall 40 of the first unshaped receptacle 38 of the first cylindrical portion 32 is relatively smooth to engage the smooth outer circumference 23 of the needle sheath of the small safety syringe 9.

The second inner circumferential wall 44 of the second unshaped receptacle 42 of the second cylindrical portion 33 includes a first sealing shoulder 56 and a first recess 58. The first recess 58 is sized to receive and lock a circumferential shoulder 27, as shown in FIG. 1, extending from the extendable, tubular sheath 22 of the medium safety syringe 10 in the plug 26. The first sealing shoulder 56 is sized to engage and seal against the outer circumference 23 of the extendable, tubular sheath 22. The purpose of the first sealing shoulder 56 and the first recess 58 is to lock the plug 26 and the medium safety syringe 10 together to prevent leakage.

Likewise, the third inner circumferential wall 48 of the third u-shaped receptacle 46 of the third cylindrical portion 34 includes a second sealing shoulder 60 and a second recess 62. The second recess 62 is sized to receive and lock the circumferential shoulder 27, as shown in FIG. 1, extending from the extendable, tubular sheath 22 of the large safety syringe 11. The second sealing shoulder 60 is sized to engage and seal against the outer circumference 23 of the extendable, tubular sheath 22. Again, the purpose of the sealing shoulder 60 and the recess 62 is to lock the plug 26 and the large safety syringe 11 together to prevent leakage.

FIG. 8 is a sectional view of a small safety syringe 9 with the extendable, tubular sheath 22 in the fully extended position. The extendable, tubular sheath 22 and the plug 26 are not engaged in this drawing. The outer circumference 23 of the extendable, tubular sheath 22 of this small safety syringe 9 does not have a shoulder 27 like the medium safety syringe 10 and the large safety syringe 11. This illustrates that safety syringes of different configurations are suitable for use with this present invention.

FIG. 9 is a sectional view of the small safety syringe 9 with the extendable, tubular sheath 22 in the fully extended position. The extendable, tubular sheath 22 and the plug 26 are now fully engaged.

FIG. 10 is a sectional view of a large safety syringe 11 with the extendable, tubular sheath 22 in the fully extended position. The extendable, tubular sheath 22 and the plug 26 are not engaged in this drawing.

FIG. 11 is a sectional view of the large safety syringe 11 with the extendable, tubular sheath 22 in the fully extended position. The extendable, tubular sheath 22 and the plug 26 are now fully engaged.

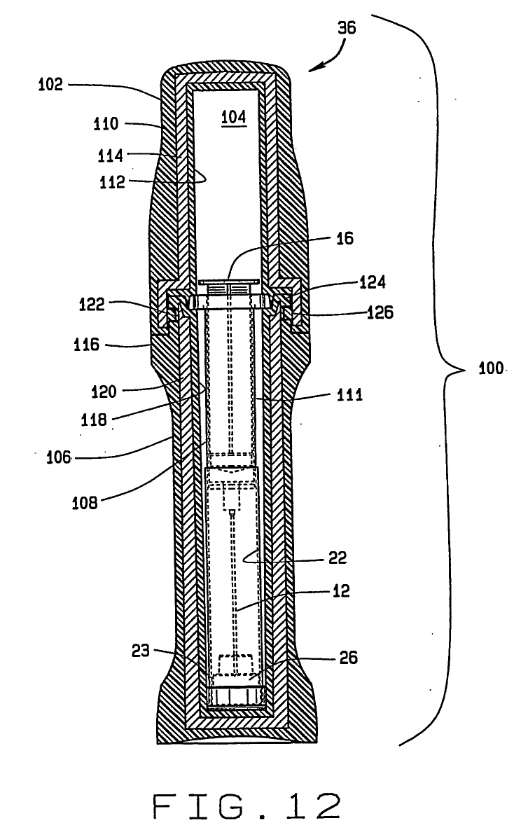

FIG. 12 is a sectional view of an assembled pharmaceutical pig 36, with the large safety syringe 11 and plug 26, as shown in FIG. 11, positioned inside the pharmaceutical pig 36. The pharmaceutical pig 36 includes a cap 102, having a first hollow center section 104, and a base 106 having a second hollow center section 108. The cap 102 and the base 106 have a twist closure, not shown. The first hollow center section 104 and the second hollow center section 108 define a hollow center section 111 for the pharmaceutical pig 36. The needle 12, the extendable, tubular sheath 22 and the plug 26 are positioned in the second hollow center section 108 of the base 106. At least a portion of the plunger 16 is positioned in the first hollow center section 104 of the cap 102.

The cap 102 has an outer shell 110, an inner shell 112 and an intermediate radiation shield 114. The base 106 has an outer shell 116, an inner shell 118 and an intermediate radiation shield 120. Preferably the outer and inner shells, 110 and 112, respectively, are formed from plastic and the intermediate radiation shield 114 is formed from lead or tungsten. A seal 122 is positioned about the neck 124 of the base 106. The seal 122 is positioned in a channel 126 of the base 106 to provide a fluid tight seal between the cap 102 and the base 106.

FIG. 13 is a sectional view of the medium safety syringe 10 with the extendable, tubular sheath 22 in the fully extended position. The extendable, tubular sheath 22 and an alternative embodiment of the plug 260 are not engaged. The plug 260 of the alternative embodiment includes an end cap 280, a cylindrical portion 300, a first flange 320 and a second flange 340. The safety syringe shown in this drawing is the same as the safety syringe shown in FIG. 1; therefore, the numerals used to describe the safety syringe in FIG. 1 are the same as those used to describe the safety syringe in this drawing. The primary difference between the plug 26 of the preferred embodiment (FIGS. 4-7) and the plug 260 of this alternative embodiment is the manner in which the plugs 26, 260 engage the extendable, tubular sheath 22. As previously described, the plug 26 of the preferred embodiment engages the outside circumference 23 of the extendable, tubular sheath 22. The plug 260 of the alternative embodiment engages the inside circumference 35 of the extendable, tubular sheath 22 as will be better seen in FIG. 14.

FIG. 14 is a sectional view of the medium safety syringe 10 of FIG. 13 with the extendable, tubular sheath 22 in the fully extended position. The extendable, tubular sheath 22 and the plug 260 are engaged. The inside circumference 35 of the open end 24 of the extendable, tubular sheath 22 engages a portion of the plug 260. The outside circumference 321 of the first flange 320 and the outside circumference 341 of the second flange 340 engage the inside circumference 35 of the open end 24 of the extendable, tubular sheath 22. The end cap 280 is mounted flush against the open end 24 to provide a liquid-tight seal. The end cap 280 preferably has a substantially similar diameter as the extendable, tubular sheath 22. The combination of the medium safety syringe 10 and the plug 26 is generally indicated by numeral 600. This will result in the medium safety syringe 10 being sealed to prevent leakage of liquid radiopharmaceuticals and/or bloodborne pathogens and reduce exposure occurring after administration of the radiopharmaceutical to the patient. Although not necessary, the needle 12 is preferably removable from the medium safety syringe 10.

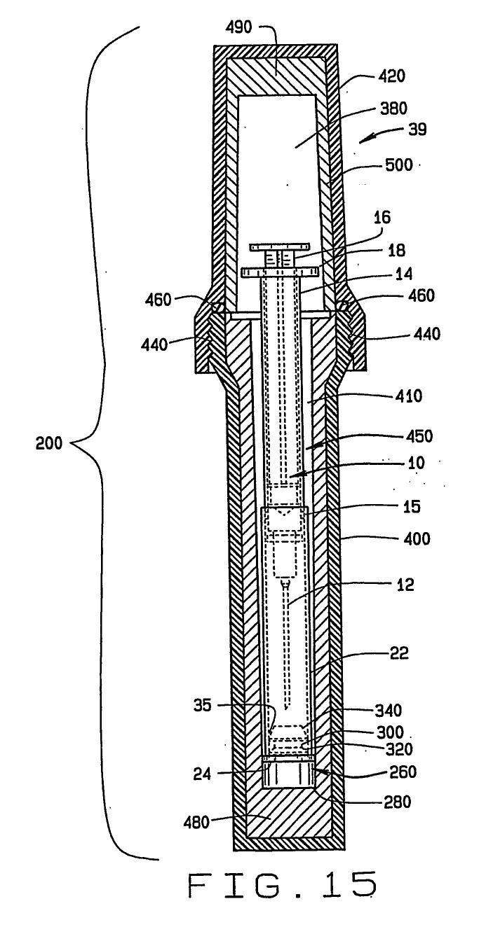

Referring now to FIG. 15, which illustrates a sectional view of an assembled pharmaceutical pig, which is generally indicated by numeral 39. The medium safety syringe 10 and the plug 260 of FIG. 14 are positioned inside the pharmaceutical pig 39. The combination of the pharmaceutical pig 39, the medium safety syringe 10 and the plug 26 is generally indicated by numeral 200.

The pharmaceutical pig 39 can hold safety syringes of different volumes of fluid, e.g., 1 cubic centimeter (0.06 cubic inches), 3 cubic centimeters (0.18 cubic inches), 5 cubic centimeters (0.31 cubic inches), 6 cubic centimeters (0.37 cubic inches), 10 cubic centimeters (0.61 cubic inches) and 12 cubic centimeters (0.73 cubic inches). Therefore, the pharmaceutical pig 39 can be built to accommodate the various sizes and volumes of safety syringes, or in the alternative, pharmaceutical pigs 39 of differing sizes can be selected to accommodate the various sizes and volumes of safety syringes. For example, one size of pharmaceutical pig 39 could accommodate the medium safety syringes 10 of 6 cubic centimeters (0.37 cubic inches) of volume and another size of pharmaceutical pig, not shown, could accommodate the 10 cubic centimeters (0.61 cubic inches) and 12 cubic centimeters (0.73 cubic inches) safety syringes.

After administration of the radiopharmaceutical to the patient, the plunger 16 may be fully depressed. The extendable, tubular sheath 22 is then moved to the fully extended position to surround the needle 12. The extendable, tubular sheath 22 is then locked and prevented from returning to the retracted position with the locking mechanism 15 by rotating the extendable, tubular sheath 22 about the barrel 14 of the medium safety syringe 10. The plug 260 is then engaged with the inner circumference 35 of the open end 24 of the extendable, tubular sheath 22. The preferred, but nonlimiting, embodiment of the plug 260 includes the cylindrical portion 300 having at least one flange extending therefrom, e.g., the first flange 320 and the second flange 340. The illustrative first flange 320 and illustrative second flange 340 preferably, but not necessarily, abut the inside circumference of the open end 24 of the extendable, tubular sheath 22. The end cap 280 preferably has a larger diameter than the cylindrical portion 300 and is mounted flush against the open end 24 of the extendable, tubular sheath 22 to provide sealing and then is placed within the pharmaceutical pig 39, as shown in FIG. 15.

The pharmaceutical pig 39 includes a cap 420, having a first hollow center section 380, and a base 400, having a second hollow center section 410. The first hollow center section 380 and the second hollow center section 410 define a hollow center section 450 for the pharmaceutical pig 39. The needle 12, extendable, tubular sheath 22 and plug 260 are positioned in the second hollow center section 410 of the base 400 of the pharmaceutical pig 39. At least a portion of the plunger 16 is positioned in the first hollow center section 380 of the cap 420. The cap 420 threadably engages the base 400 through a series of threaded grooves generally indicated by numeral 440 to secure the cap 420 to the top of the base 400. There is an o-ring 460 that is under compression that provides a fluid-tight seal between the cap 420 and the base 400. The base 400 includes a radiation shield 480 and the cap includes a radiation shield 490. Preferably, the pharmaceutical pig 39 is a container that has been approved by the United States Department of Transportation.

Method of Use for the Combination of Safety Syringe and Plug 26

Although the combination safety syringe 9, 10, 11 and plug 26, as shown in FIGS. 2-3 and 8-11 indicated by numeral 60 can be utilized in any application of medicine, it is especially applicable to nuclear medicine. For example, with nuclear medicine, a prescription is provided to a radiopharmacy with the pharmacist filing the safety syringe 9, 10 or 11 with the liquid radiopharmaceutical in accordance with the prescription. The filled safety syringe 9, 10 or 11 is assayed by measuring the activity of the liquid radiopharmaceutical in the safety syringe 9, 10 or 11 with a dose calibrator to verify compliance with the prescription. The liquid radiopharmaceutical is then administered to the patient by snuggly fitting the safety syringe 9, 10 or 11 into a radiation/injection shield (not shown). This process includes removing the removable needle cover 20, as shown in FIG. 1 and inserting the needle 12 into a needle port or the patient and depressing the plunger 16. The needle 12 can also be removed from the safety syringe 9, 10 or 11 and the safety syringe 9, 10 or 11 can be connected to a needleless injection port. The plunger 16 does not necessarily need to be depressed completely to the finger grips 18 although the full dose is often given to the patient and the grips 18 are fully depressed.

After administration of the radiopharmaceutical, as shown in FIGS. 2, 8 and 10, the extendable, tubular sheath 22 is then moved to the fully extended position to surround the needle 12 and is then locked and prevented from returning to the retracted position with the locking mechanism 15 by rotating the extendable, tubular sheath 22 about the barrel 14 of the safety syringe 9, 10, 11.

The plug 26 is then aligned with the open end 24 of the extendable, tubular sheath 22. As shown in FIG. 8, the open end 24 of the extendable, tubular sheath 22 of the small safety syringe 9 is positioned over the plug 26 so that the first cylindrical portion 32 can receive and seal with the outer circumference 23 of the extendable, tubular sheath 22. As shown in FIGS. 2 and 3, the medium safety syringe 10 is also positioned over the plug 26 so the second cylindrical portion 33 can receive and seal with the outer circumference 23 of the extendable, tubular sheath 22. As shown in FIGS. 10 and 11, the large safety syringe 11 is likewise positioned over the plug 26 so the third cylindrical portion 34 can receive and seal with the outer circumference 23 of the extendable, tubular sheath 22. Regardless of the size of the safety syringe 9, 10, 11, the plug 26 completely seals the needle 12 in a leakproof relationship to prevent any liquid radiopharmaceutical and/or bloodborne pathogen from leaking out of the safety syringe 9, 10, 11.

This is believed to provide compliance with the revised Bloodborne Pathogens Standard (29 C.F.R. Sectional 1910.1030) promulgated by the Occupational Safety and Health Administration by fully meeting their definition of a “safe needle” by providing a complete and total barrier from the needle 12 that remains safe after disposal. However the safety syringe 9, 10, 11 utilized must be one that: provides a barrier from the needle 12; allows the hands of the healthcare provider to remain behind the needle 12; has the extendable, tubular sheath 22 function as part of the safety syringe 9, 10, 11 and not as an accessory; and is simple to operate.

Furthermore, it is also believed that the combination safety syringe 9, 10, 11 and plug 26 as indicated by numeral 60 also meets the criteria for a “safety syringe” as defined by the Council on Radionuclides and Radiopharmaceuticals by providing a safety syringe 9, 10, 11 that does not leak at any time as long as the selected safety syringe 9, 10, 11: fits snuggly within a radiation/injection shield (not shown); can be operated without displacing the radiation/injection shield; has a removable needle 12; and the safety syringe 9, 10, 11 can fit within a shipping container approved by the United States Department of Transportation.

Moreover, the combination safety syringe 9, 10, 11 and plug 26 as indicated by numeral 60 is believed to comply with the revised Bloodborne Pathogens Standard (29 C.F.R. Sectional 1910.1030(d)(2)) promulgated by the Occupational Safety and Health Administration by fully meeting their definition of a “sharps container” by providing a container that is: puncture resistant; capable of labeling or color-coding; leakproof on the sides and bottom; and does not require a healthcare provider to reach by hand into the container where the sharp has been placed.

Method of Use for the Combination of Pharmaceutical Pig 36, Safety Syringe and Plug 26

Although the combination of pharmaceutical pig 36, safety syringe 9, 10 and 11 and plug 26, as shown in FIG. 12 and indicated by numeral 100, can be utilized in any application of medicine, it is especially applicable to nuclear medicine. For example, with nuclear medicine, a prescription is provided to a radiopharmacy with the pharmacist filling the safety syringe 9, 10, 11 with the liquid radiopharmaceutical in accordance with the prescription. The filled safety syringe 9, 10, 11 is assayed by measuring the activity of the liquid radiopharmaceutical in the safety syringe 9, 10, 11 with a dose calibrator to verify compliance with the prescription. The filled safety syringe 9, 10, 11 is then placed in the base 106 of the pharmaceutical pig 36. The cap 102 is then threaded onto the base 106 utilizing the threaded grooves, not shown.

The pharmaceutical pig 36 is then closed and wipe tested for unwanted nuclear activity. If the pharmaceutical pig 36 passes the wipe test, it is placed in a delivery container (not shown). A delivery container typically has an interior padding of rubber foam. A plurality of receptacles are formed in the rubber foam and each is shaped to receive a pharmaceutical pig 36. Several pharmaceutical pigs 36 may be placed in a single delivery container. Before leaving the radiopharmacy, the delivery container and the pharmaceutical pigs 36 are wipe tested and surveyed. If the delivery container passes, a United States Department of Transportation “DOT” label is affixed to the outside of the delivery container. The DOT label contains the radioactivity symbol and the word “Radioactive”. The delivery container is delivered to a medical treatment facility.

Upon receipt of a delivery container by the medical treatment facility, the pharmaceutical pig 36 is then opened and typically, the filled safety syringe 9, 10, 11 is placed in an injection sheath (not shown). The liquid radiopharmaceutical is then administered to the patient. This process includes removing the removable needle cover 20, as shown in FIG. 1, and depressing the plunger 16. The plunger 16 does not necessarily need to be depressed completely to the finger grips 18, but is often fully depressed to administer a full dose.

As shown in FIGS. 2, 8 and 10 the extendable, tubular sheath 22 is then moved to the fully extended position to surround the needle 12 then locked and prevented from returning to the retracted position with the locking mechanism 15 by rotating the extendable, tubular sheath 22 about the barrel 14 of the safety syringe 9, 10, 11.

A first method is to align the plug 26 with the open end 24 of the extendable, tubular sheath 22. As shown in FIG. 8, the open end 24 of the extendable, tubular sheath 22 of the small safety syringe 9 is positioned over the plug 26 so that the first cylindrical portion 32 can receive and seal with the outer circumference 23 of the extendable, tubular sheath 22. As shown in FIGS. 2 and 3, the medium safety syringe 10 is also positioned over the plug 26 so the second cylindrical portion 33 can receive and seal with the outer circumference 23 of the extendable, tubular sheath 22. As shown in FIGS. 10 and 11, the large safety syringe 11 is likewise positioned over the plug 26 so the third cylindrical portion 34 can receive and seal with the outer circumference 23 of the extendable, tubular sheath 22. Preferably, this is done in a manner where the health care provider can keep his or her hands behind the extendable, tubular sheath 22 and not in front of the needle 12. This completely seals the needle 12 in a fluid-tight relationship to prevent any liquid radiopharmaceutical and/or bloodborne pathogen from leaking out of the safety syringe 9, 10, 11.

A second and preferred method is to insert the plug 26 into the base 106 of the pharmaceutical pig 36. The end cap 28 will then rest against the inner shell 118 of the base 40 with the first, second and third cylindrical portions 32, 33 and 34, respectively, pointing upward toward the cap 102 of the pharmaceutical pig 36. The used safety syringe 10 is then inserted into the base 106 the pharmaceutical pig 36 so that the outer circumference 23 of the extendable, tubular sheath 22 engages the plug 26. The plug 26 is then positioned flush against the open end 24 of the extendable, tubular sheath 22. This completely seals the needle 12 in a fluid-tight relationship to prevent any liquid radiopharmaceuticals and/or bloodborne pathogens from leaking out of the safety syringe 9, 10, 11 as well as completely eliminating any opportunity for this used safety syringe 9, 10, 11 to inadvertently stick another patient. This second method is preferred since the healthcare professional does not have to get anywhere near the tip of the needle 12 for the safety syringe 9, 10, 11.

After all the pharmaceutical pigs 36 have been opened and the liquid radiopharmaceuticals have been administered, the delivery case with the pharmaceutical pigs 36 and used safety syringes 9, 10, 11 are then returned to the radiopharmacy. The used safety syringes 9, 10, 11 are removed from the pharmaceutical pig 36 and placed in a disposal bin. The pharmaceutical pig 36 is then ready to be reused.

This is believed to provide full compliance with the revised Bloodborne Pathogens Standard (29 C.F.R. Sectional 1910.1030) promulgated by the Occupational Safety and Health Administration by fully meeting their definition of a “safe needle” by providing a complete and total barrier from the needle 12 that remains safe after disposal by sealing the safety syringe 9, 10, 11 with both the plug 26 and the pharmaceutical pig 36. However, the safety syringe 9, 10, 11 selected must be one that: provides a barrier from the needle 12; allows the hands of the healthcare provider to remain behind the needle 12; has the extendable, tubular sheath 22 that functions as part of the safety syringe 9, 10, 11 and not as an accessory, and is simple to operate.

Furthermore, it is believed that the combination safety syringe 9, 10, 11, plug 26 and pharmaceutical pig 36 as indicated by numeral 100 also meets the criteria for a safety syringe as defined by the Council on Radionuclides and Radiopharmaceuticals by providing a safety syringe 9, 10, 11 that does not leak at any time as long as the selected safety syringe 9, 10, 11: fits snuggly within a radiation/injection shield (not shown); can be operated without displacing the radiation/injection shield; has a removable needle 12; and can fit within the pharmaceutical pig 36, where the pharmaceutical pig 36 has been approved by the U.S. Department of Transportation.

Moreover, the combination safety syringe, plug 26 and pharmaceutical pig 36 as indicated by numeral 100 is believed to comply with the revised Bloodborne Pathogens Standard (29 C.F.R. Sectional 1910.1030(d)(2)) promulgated by the Occupational Safety and Health Administration by fully meeting their definition of a “sharps container” by providing a container that is: puncture resistant; capable of being labeled or color-coded; leakproof on the sides and bottom; and does not require a healthcare provider to reach by hand into the container where the sharp has been placed.

A preferred embodiment of a plug is generally indicated by numeral 26 in FIGS. 2-3, 8-11. This plug 26 can be of a wide variety of shapes and sizes, which operate to close the open end 24 of the extendable, tubular sheath 22.

Method of Use for the Combination of Safety Syringe and Plug 260

The combination safety syringe 9, 10, 11 and plug 260 as shown in FIGS. 13 and 14 and as indicated by numeral 600 can be utilized in any application of medicine. However, this combination is especially applicable to nuclear medicine. For example, with nuclear medicine, a prescription is provided to a radiopharmacy with the pharmacist filing the safety syringe 9, 10, 11 with the liquid radiopharmaceutical in accordance with the prescription. As previously discussed, the size of the safety syringe 9, 10, 11 is typically dependent on the prescription. The filled safety syringe 9, 10, 11 is assayed by measuring the activity of the liquid radiopharmaceutical in the safety syringe 9, 10, 11 with a dose calibrator to verify compliance with the prescription. The safety syringe 9, 10, 11 is then put into a radiation/injection shield (not shown). The liquid radiopharmaceutical is then administered to the patient by depressing the plunger 16. The plunger 16 does not necessarily need to be depressed completely to the finger grips 18 but is often fully depressed to administer a full dose.

The extendable, tubular sheath 22 is then moved to the fully extended position to surround the needle 12 and is then locked and prevented from returning to the retracted position with the locking mechanism 15 by rotating the extendable, tubular sheath 22 about the barrel 14 of the safety syringe 9, 10, 11.

The plug 260 is then aligned with the open end 24 of the extendable, tubular sheath 22 as shown in FIG. 13. The open end 24 of the extendable, tubular sheath 22 is positioned over the cylindrical portion 300 of the plug 260 so that the first flange 320 and second flange 340 create a seal against the inside circumference 35 of the extendable, tubular sheath 22. The end cap 280 is then positioned flush against the open end 24 of the extendable, tubular sheath 22. As required by regulation, this is done in a manner where the health care provider can keep his or her hands behind the extendable, tubular sheath 22 and not in front of the needle 12. This completely seals the needle 12 in a leakproof relationship to prevent any liquid radiopharmaceutical and/or bloodborne pathogen from leaking out of the safety syringe 9, 10, 11.

This is believed to provide compliance with the revised Bloodborne Pathogens Standard (29 C.F.R. Sectional 1910.1030) promulgated by the Occupational Safety and Health Administration by fully meeting their definition of a “safe needle” by providing a complete and total barrier from the needle 12 that remains safe after disposal. However the safety syringe 9, 10, 11 utilized must be one that: provides a barrier from the needle 12; allows the hands of the healthcare provider to remain behind the needle 12; has the extendable, tubular sheath 22 function as part of the safety syringe 9, 10, 11 and not as an accessory; and is simple to operate.

Furthermore, it is also believed that the combination safety syringe 9, 10, 11 and plug 260 as indicated by numeral 600 also meets the criteria for a “safety syringe” as defined by the Council on Radionuclides and Radiopharmaceuticals by providing a safety syringe 9, 10, 11 that does not leak at any time as long as the selected safety syringe: fits snuggly within a radiation/injection shield (not shown); can be operated without displacing the radiation/injection shield; has a removable needle 12; and the safety syringe 9, 10, 11 can fit within a shipping container approved by the United States Department of Transportation.

Moreover, the combination safety syringe 9, 10, 11 and plug 260 as indicated by numeral 600 is believed to comply with the revised Bloodborne Pathogens Standard (29 C.F.R. Section 1910.1030 (d)(2)) promulgated by the Occupational Safety and Health Administration by fully meeting their definition of a “sharps container” by providing a container that is: puncture resistant; capable of labeling or color-coding; leakproof on the sides and bottom; and does not require a healthcare provider to reach by hand into the container where the sharp has been placed.

Method of Use for the Combination of Pharmaceutical Pig 39, Safety Syringe and Plug 260

Although the combination of pharmaceutical pig 39, safety syringe 9, 10, 11 and plug 260, as shown in FIG. 15 and indicated by numeral 200, can be utilized in any application of medicine, it is especially applicable to nuclear medicine. For example, with nuclear medicine, a prescription is provided to a radiopharmacy with the pharmacist filling the safety syringe with the liquid radiopharmaceutical in accordance with the prescription. Depending on the prescription, the pharmacist may select a medium safety syringe 10, a small safety syringe 9 or a large safety syringe 11. For purposes of this example, the pharmacist has selected a medium size syringe 10. The filled safety syringe is assayed by measuring the activity of the liquid radiopharmaceutical in the safety syringe with a dose calibrator to verify compliance with the prescription. The filled medium safety syringe 10 is then placed in the base 400 of the pharmaceutical pig 39. The cap 420 is then threaded onto the base 400 utilizing the threaded grooves 440 with the filled safety syringe 10 secured within the second hollow center section 410 of the base 400. The pharmaceutical pig 39 is then closed and wipe tested for unwanted nuclear activity. If the pharmaceutical pig 39 passes the wipe test, it is placed in a delivery container (not shown). A delivery container typically has an interior padding of rubber foam. A plurality of receptacles are formed in the rubber foam and each is shaped to receive a pharmaceutical pig 39. Several pharmaceutical pigs 39 may be placed in a single delivery container. Before leaving the radiopharmacy, the delivery container and the pharmaceutical pigs 39 are wipe tested and surveyed. If the delivery container passes, a United States Department of Transportation “DOT” label is affixed to the outside of the delivery container. The DOT label contains the radioactivity symbol and the word “Radioactive”. The delivery container is delivered to a medical treatment facility.

Upon receipt of a delivery container by the medical treatment facility, the pharmaceutical pig 39 is then opened and typically, the filled safety syringe 10 is placed in an injection sheath (not shown). The liquid radiopharmaceutical is then administered by depressing the plunger 16. The plunger 16 does not necessarily need to be depressed completely to the finger grips 18 but is often fully depressed to administer a full dose of the radiopharmaceutical.

As shown in FIG. 13, the extendable, tubular sheath 22 is then moved to the fully extended position to surround the needle 12 then locked and prevented from returning to the retracted position with the locking mechanism 15 by rotating the extendable, tubular sheath 22 about the barrel 14 of the safety syringe 10.

A first method is to align the plug 260 with the open end 24 of the extendable, tubular sheath 22. As shown in FIG. 14, the cylindrical portion 300 of the plug 260 is then engaged with the open end 24 of the extendable, tubular sheath 22 with the first flange 320 and second flange 340 creating a seal against the inside circumference 35 of the extendable, tubular sheath 22. The end cap 280 is then positioned flush against the open end 24 of the extendable, tubular sheath 22. Preferably, this is done in a manner where the health care provider can keep his or her hands behind the extendable, tubular sheath 22 and not in front of the needle 12. This completely seals the needle 12 in a fluid-tight relationship to prevent any liquid radiopharmaceutical and/or bloodborne pathogen from leaking out of the safety syringe 10.

A second and preferred method is to insert the plug 260 into the base 400 of the pharmaceutical pig 39. The end cap 280 will then rest against the radiation shield 480 of the base 400 with the cylindrical portion 300 pointing upward toward the cap 420 of the pharmaceutical pig 39. The used safety syringe 10 is then inserted into the base 400 of the pharmaceutical pig 39 so that the open end 24 of the extendable, tubular sheath 22 encloses the cylindrical portion 300 of the plug 260 so that the first flange 320 and second flange 340 that extend outwardly from the cylindrical portion 300 abut and form a seal against the inside circumference 35 of the extendable, tubular sheath 22 through the open end 24. The end cap 280 is then positioned flush against the open end 24 of the extendable, tubular sheath 22. This completely seals the needle 12 in a fluid-tight relationship to prevent any liquid radiopharmaceuticals and/or bloodborne pathogens from leaking out of the safety syringe 10 as well as completely eliminating any opportunity for this used safety syringe 10 to inadvertently stick another patient. This second method is preferred since the healthcare professional does not have to get anywhere near the tip of the needle 12 for the safety syringe 10.

After all the pharmaceutical pigs 39 have been opened and the liquid radiopharmaceuticals have been administered, the delivery case with the pharmaceutical pigs 39 and used safety syringes 10 are then returned to the radiopharmacy. The used safety syringes 10 are removed from the pharmaceutical pigs 39 and placed in a disposal bin. The pharmaceutical pig 39 is then ready to be reused.

This is believed to provide full compliance with the revised Bloodborne Pathogens Standard (29 C.F.R. Sectional 1910.1030) promulgated by the Occupational Safety and Health Administration by fully meeting their definition of a “safe needle” by providing a complete and total barrier from the needle 12 that remains safe after disposal by sealing the safety syringe 10 with both the plug 260 and the pharmaceutical pig 39. However, the safety syringe 10 selected must be one that: provides a barrier from the needle 12; allows the hands of the healthcare provider to remain behind the needle 12; has the extendable, tubular sheath 22 that functions as part of the safety syringe 10 and not as an accessory; and is simple to operate.

Furthermore, it is believed that the combination safety syringe 10, plug 260 and pharmaceutical pig 39 as indicated by numeral 200 also meets the criteria for a safety syringe 10 as defined by the Council on Radionuclides and Radiopharmaceuticals by providing a safety syringe 10 that does not leak at any time as long as the selected safety syringe 10: fits snuggly within a radiation/injection shield (not shown); can be operated without displacing the radiation/injection shield; has a removable needle 12; and can fit within the pharmaceutical pig 39, where the pharmaceutical pig 39 has been approved by the U.S. Department of Transportation.

Moreover, the combination safety syringe 10, plug 260 and pharmaceutical pig 39 as indicated by numeral 200 is believed to comply with the revised Bloodborne Pathogens Standard (29 C.F.R. Sectional 1910.1030(d)(2)) promulgated by the Occupational Safety and Health Administration by fully meeting their definition of a “sharps container” by providing a container that is: puncture resistant; capable of being labeled or color-coded; leakproof on the sides and bottom; and does not require a healthcare provider to reach by hand into the container where the sharp has been placed.

Although a preferred embodiment of the safety syringe sealing system and method of use has been illustrated in the accompanying Drawings and described in the foregoing Detailed Description, it is understood that the invention is not limited to the embodiment disclosed, but is capable of numerous rearrangements, modifications and substitutions without departing from the spirit for the invention as set forth and defined by the following claims.

1. A safety syringe sealing system, the system comprising:
a safety syringe having a barrel, a needle, a plunger and a needle sheath, wherein the needle sheath is movable relative to the barrel and the needle between a retracted position where the needle is exposed, to an extended position where the needle is protected, the needle sheath having an open end defined therein; and
a plug that is engageable with the needle sheath at the open end thereof when the needle sheath is in the extended position.

2. The system of claim 1, wherein the needle sheath is tubular.

3. The system of claim 1, wherein the needle sheath, when in the extended position, is locked and prevented from returning to the retracted position.

4. The system of claim 1, wherein the needle sheath includes means for rotating the needle sheath about the barrel of the safety syringe to lock the needle sheath in the extended position.

5. The system of claim 1, wherein the plug includes at least one receptacle to accommodate a portion of the needle sheath.

6. The system of claim 1, wherein the plug includes at least one flange that extends radially outward therefrom and that contacts an inner surface of the needle sheath when the plug is engaged with the needle sheath.

7. The system of claim 1, further comprising a pharmaceutical pig having a base and a cap, the base and cap each having a hollow center section that is capable of accommodating a portion of the safety syringe.

8-12. (canceled)

13. The system of claim 7, wherein the base and cap of the pharmaceutical pig interact to seal the safety syringe within the pharmaceutical pig.

14. A method of using a safety syringe system, the method comprising:
moving a needle sheath of a safety syringe from a retracted position where a needle of the safety syringe is exposed, to an extended position where the needle is protected, the needle sheath having an open end defined therein; and
plugging the open end of the needle sheath when the needle sheath is in the extended position.

15. The method of claim 14, wherein the moving of the needle sheath includes preventing the needle sheath from returning to the retracted position.

16. The method of claim 14, wherein the plugging of the open end of the needle sheath includes engaging a plug around an outer circumference of the needle sheath proximate the open end thereof when the needle sheath is in the extended position.

17. (canceled)

18. The method of claim 14, wherein the plugging of the open end of the needle sheath includes engaging a plug, having a cylindrical portion and at least one flange extending radially therefrom, with the open end of the needle sheath.

19. The method of claim 14, wherein the plugging of the open end of the needle sheath includes engaging a plug, having at least one unshaped receptacle, with an outside circumference of the needle sheath.

20. A method of using a safety syringe system, the method comprising:
inserting a plug in a hollow center section of a base of a pharmaceutical pig having a cap;
moving a needle sheath of a safety syringe from a retracted position where a needle of the safety syringe is exposed to an extended position where the needle is protected, the needle sheath having an open end defined therein through which the needle passes during the moving of the needle sheath from the retracted position to the extended position;
inserting the safety syringe into the hollow center section of the base of the pharmaceutical pig, wherein the inserting of the safety syringe includes engaging the sheath plug in the pharmaceutical pig with the needle sheath at the open end thereof to plug the open end of the needle sheath, the needle sheath being in the extended position during the engaging of the sheath plug with the needle sheath.

21. The method of claim 20, further including locking the needle sheath when the needle sheath is in the extended position to prevent the needle sheath from returning to the retracted position from the extended position.

22. The method of claim 21, wherein the locking of the needle sheath includes rotating the needle sheath about the barrel of the safety syringe.

23. The method of claim 22, further comprising sealing the base and a cap of the pharmaceutical pig together to contain the safety syringe within the pharmaceutical pig.

24. The method of claim 20, wherein the engaging of the sheath plug with the needle sheath includes engaging the sheath plug with an outside circumference of the needle sheath.

25-31. (canceled)

32. The method of claim 20, wherein the engaging of the sheath plug with the needle sheath includes contacting the needle sheath with at least one flange that extends radially from the sheath plug.

33. The method of claim 20, wherein the sheath plug includes at least one unshaped receptacle.
